(19) United States
(12) Patent Application Publication
(10) Pub. No.: US 2008/0051351 A1
(43) Pub. Date: Feb. 28, 2008

(54) Substances, Compositions, and Methods for Treating Alopecia

(76) Inventor: Carlo Ghisalberti, Milano (IT)

Correspondence Address: OHLANDT, GREELEY, RUGGIERO & PERLE, LLP, ONE LANDMARK SQUARE, 10TH FLOOR, STAMFORD, CT, 06901, US

(21) Appl. No.: 11/884,716

(22) PCT Filed: Feb. 21, 2006
(86) PCT No.: PCT/EP06/60131

§ 371 Date: Aug. 20, 2007

(30) Foreign Application Priority Data
Feb. 21, 2005 (IT) MI2005A000262

(51) Int. Cl. A61K 31/702; A61K 8/60; A61P 17/14; A61Q 7/00

(52) U.S. Cl. 514/25

(57) ABSTRACT

Substances, compositions and methods for hair treatment based on ultra-low molecular weight hyaluronic acid oligomers, possibly combined with trichogenic substances, useful for preventing hair loss and favouring its regrowth in subjects affected by androgenetic alopecia, alopecia areata, alopecia mucinosa and related disorders.

### FIELD OF THE INVENTION

The present invention relates to compositions and methods for hair treatment based on ultra-low molecular weight hyaluronic acid oligomers, used alone or in combination with other therapeutic substances; the compositions thus obtained are useful for preventing hair loss and favouring its regrowth, particularly in subjects affected by androgenetic alopecia, alopecia areata, alopecia mucinosa and related disorders.

### PRIOR ART

Common alopecia is described as androgenetic in that a critical role can be attributed to active testosterone metabolites and the individual's predisposition to dihydrotestosterone action on the pilosebaceous unit. Indeed, current pharmacological therapies are of anti-androgenetic type, such as those based on finasteride for example, or topical applications of anti-androgens of plant—or synthetic origin.

Alopecia areata is considered instead to be a disease of various etiologies. In the autoimmune component, cells of the immune system aggregate around follicles and prevent the production of hair. Patients frequently exhibit a neurotic behaviour leading to the belief that there is a psychosomatic component. Actually stress is an important precipitating factor in alopecia areata but to date a statistical rather than etiopathogenetic correlation has been demonstrated.

Depending on the extent of hair loss, alopecia areata can be classified as alopecia totalis (AT) which involves the whole scalp, and as alopecia universalis (AU) which involves all body hairs.

Besides the androgenetic and immunological issues, the vascular components are also a critical factor in alopecia, as described by Stenn & Paus “Controls of Hair Follicle Cycling”, Physiological Reviews, 81(1), 449-494, 2001.

Follicles in the telogen phase have lower perfusion requirements than in the anagen phase whose follicles appear more vascularized than those in telogen. Both fibroblasts and papillary keratinocytes produce Vascular Endothelial Growth Factor (VEGF), while the synthesis of the endothelial DNA primarily occurs in the papillae of follicles in anagen IV.

The vascular component in alopecia has until now been largely underestimated, despite the study of Goldman C K, Tsai J C, Soroceanu L, Gillespie G Y. “Loss of vascular endothelial growth factor in human alopecia hair follicles”, J Invest Dermatol 104: 18S-20S, 1995 provided clear evidences of scarce and inadequate VEGF expression in the follicles of alopecia subjects.

Hyaluronic acid (HA) is a fundamental component of extracellular matrix. In fact it plays a key physiological role within a variety of biological tissues, such as skin, tendons, muscles and cartilage, whereby it provides hydration and lubrication as well as regulates cell migration, function and differentiation.

Moreover HA represents an important therapeutic tool in several research articles and patents thereby reporting various useful derivatives and/or its use as pharmacological carrier, see Prestwich G D, “Biomaterials from Chemically Modified Hyaluronan”, Glycoforum (Mar. 29, 2001).

There are two main approaches for the formation of HA derivatives: cross-linking with bi-functional reagents, and chemical modification with mono-functional reagents which generally react on functional moyeties: acetamide, carboxy and hydroxy groups.

Most attention is directed towards modifying carboxy and hydroxy groups in particular. For example the esterification of carboxyls is described in EP216453 with partial or total esterification of COOH by mono-functional organic halides; amidation reactions affording water insoluble HA gel are described in U.S. Pat. No. 4,937,270.

There are also numerous references to modifications of hydroxy groups. For example, U.S. Pat. No. 5,679,657 describes the sodium salts of HA with 2,6-3,6 hydroxyl groups of each disaccharide unit converted into acetyls, the product proving to be soluble in 90% aq. ethanol. The complete esterification of HA carboxyl and hydroxy groups is cited by Khan in Carbohydrate Research (1998) 306, 137-146, who describes the preparation of peracetylated derivatives of the HA benzyl ester. Included among the fundamental characteristics of HA are its antiangiogenic properties, for example as claimed in WO9423725, and its antiproliferative properties also common to both HA and its derivatives, for example as reported in WO9823648, and specifically referred to the butyric esters of HA sodium salt. The HA in the cited examples and in others reported in literature are polysaccharides of molecular weight from 20000 to 2 million, daltons i.e. having from 50 to 5000 disaccharide units per molecule.

The oligosaccharides obtained by the depolymerization of hyaluronic acid (OHA) are know and proposed in various therapeutic applications, such as in WO0204471 and EP1300412 for the use as anti-ulcer and antineoplastic agent; or in WO04003545 for the use in oncology in drug-resistant patients.

The angiogenic activity of OHA is described by D. C. West (Science 228:1324-1326, 1985) or by R. Deed (Int J. Cancer. 10; 71(2):251-6, 1997); conversely, other authors indicate their activity in combating tumors (U.S. Pat. No. 5,902,795). In EP0295092 the use of oligosaccharides containing from 7 to 50 or from 7 to 25 monosaccharide units are claimed for the treatment of alopecia. Nonetheless the aforesaid works have not lead to any commercial OHA-based anti-alopecia product.

The present invention intends to provide products with improved activity for combating hair loss and favouring its regrowth. A further scope of the invention is to identify possible synergies among the derivatives of hyaluronic acid and other trichogenic agent, with the aim to further increase the activity thereof.

### SUMMARY

The present invention relates to compositions of ultra-low molecular weight hyaluronic acid oligomers (herein defined as “UL-OHA”), useful for preparing dermatological products suitable for combating hair loss and favouring its regrowth. Such products show an activity unexpectedly greater than known compounds; moreover they are able to synergize with various compounds of trichogenous activity to provide compositions with enhanced activity.

### DETAILED DESCRIPTION OF THE INVENTION

The “ultra-low molecular weight hyaluronic acid oligomers” (UL-OHA) of the present invention are the result of the partial depolymerization of hyaluronic acid: they are oligomers formed by altered monosaccharide units of D-glucuronic acid and N-acetyl-glucosamine, and containing from 2 to 6 of said monosaccharides. Specific UL-OHA according to the invention contain 2,3,4,5 or 6 of said monosaccharides.

The techniques of HA depolymerization are known, for example from EP1300412. Enzymatic methods are preferably used, wherein the starting HA is treated with hyaluronidase in suitable quantities (e.g. 4-8 IU/mg hyaluronic acid) in a buffered solution at pH between 4 and 5, temperature between 25 and 40° C., for a period between 1 and 8 hours. Depolymerization progress can be monitored for example by TLC; the UL-OHA fraction can therefore be separated and recovered from the crude reaction mixture, e.g. by elution through an ion exchange column. The starting HA to be depolymerized can be any currently available HA form; for example from animal tissues (e.g. cockerel crests) or, even preferably, from the fermentation of broth of HA producing microbial strains.

The possible salts of the aforesaid UL-OHA are formed with physiologically acceptable acidic or basic counterions; exemplary basic counterions are quaternary ammonium salts e.g. tetrabutylammonium, or earthly-alkaline and alkaline cations such as sodium, potassium, magnesium, lithium; exemplary acidic counterions are hydrochloric, tartaric, malonic, fumaric, pamoic acid etc.

Salification is undertaken according to known methodology, preferably starting from an already depolymerised product and treating it with a suitable provider of the counterion required for salt formation.

Exemplary esters of the aforesaid UL-OHA are methyl, ethyl, benzyl, ethoxycarbonylmethyl ester etc.; they are formed on one or more hyaluronic acid—COOH groups present in the aforesaid oligomers. Other possible esters are those formed on one or more —OH groups of hyaluronic acid and/or N-acetylglucosamine. In both cases, esterification is carried out according to known methods, preferably by treating the already depolymerised product with the suitable acid or alcohol, depending on each case, needed for ester formation. Esters of the —COOH group can also be obtained by prior salification of —COOH with a suitable quaternary ammonium salt followed by treatment with a suitable compound of formula R—X where X is a halogen and R is the alkyl portion of the required ester.

The compositions containing the active principles of the invention can be conceived and utilized in various administration forms, proving useful for combating hair loss and/or favouring its regrowth, in particular for treating androgenetic alopecia, alopecia areata, alopecia mucinosa and related disorders. Examples of related disorders are anagen and telogen effluvium.

For topical use an optimal concentration of the active principles of the invention is from 0.0001% to 10%, preferably from 0.01% to 1% of the composition by weight. Dosage depends on the administration form, preferably local application such as topical and transdermal administration.

Treatment duration depends on the pathology under consideration and the desired effect. For example the application period ranges from 1 to 50 weeks with a frequency of 3 to 14 times per week, depending on the pathological factors of the patient. The patient is preferably a male subject.

The compositions for topical use include creams, ointments, lotions, gels, aqueous emulsions, unguents, milks, lipogels and other suitable hydroglycolic, hydroalcoholic and biphasic O/W and W/O systems, thereby packaged in appropriate containers according to viscosity and mode of application. Various carriers can be used, including water, alcohol and other physiological solvents, or with oils and lipids combined with water by means of emulsifiers or surfactants.

Topical treatment can also be carried out with solid carriers suited for local application, e.g. in occlusive forms such as a bandage, sponge or patch.

Particularly useful in the compositions is the presence of trichogenic agents which can contribute to the revascularization action of UL-OHA and, in some cases, synergise them in the hair regrowth activity.

The “trichogenic agent” are defined by Andrea Marliani in “Trichology—diagnostics and therapy”, II electronic ed., Electronic Editions “Tricoltalia” (Florence), May 1997, particularly the chapter “Review of endocrine biochemistry—Current and emerging therapies”.

They can be briefly classified according to their mechanism of action into:
inhibitors of testosterone 5-alpha-reductase
androgen transport antagonists
vasokinetics and vasodilators
phosphodiesterase inhibitors
retinoids

The “inhibitors of testosterone 5-alpha-reductase” slow down the formation of dihydroxytestosterone (DHT) by the direct inhibition of the specific conversion enzyme. Illustrative examples of testosterone 5-alpha-reductase inhibitors of steroidal structure include: finasteride, dutasteride, N-t-butyl-3-oxo-4-aza-5α-androst-1-ene-17β-carboxamide, estrogens (e.g. estrone, estrone sulfate, 17-alpha-estradiol), and progesterone; illustrative examples of non-steroidal testosterone 5-alpha-reductase inhibitors include: zinc salts and complexes, polyunsaturated fatty acids (e.g. ximenic, gamma-linoleic, alpha-linoleic, linolenic, docosahexaenoic, eicosapentaenoic, eicosahexaenoic acids), pyridoxine, and azelaic acid.

The “androgen transport antagonists” are specific agents able to inhibit the binding between active steroidal hormones, such as DHT and its precursors, and their transport receptors. They may operate with a competitive mechanism directly on transport receptors of DHT and precursors, or may by compete with aldosterone at the level of cytoplasmic receptor sites with the formation of inactive complexes, or may decrease the testosterone availability by binding it to the sex hormone binding globulins (SHBG). Illustrative examples of such antagonists include: spironolactone, flutamide, cyproterone acetate, potassium canrenoate, cimetidine, phytosterols (e.g. beta sitosterol, campesterol, stigmasterol), lignans (e.g. (−)-3,4-divanillyltetrahydrofuran, (−)-matairesinol, (−)-secoisolariciresinol, (±)-enterolactone, (±)-enterodiol, nordihydroguaiaretic acid); and thyroid hormones (e.g. L-thyroxine, triiodotyrosine, D-thyroxine). Within the latter sub-class D-thyroxine (synthetic dextrorotary form of thyroxine) is preferred in that it lacks a hormonal effect while retaining the ability to increase the affinity of testosterone for SHBG.

The “vasokinetics and vasodilators” as here defined is a large class of substances that increase blood flow into the hair follicle, being of particular importance to sustain the anagen phase, and also acting as antifibrotic agent at the perifollicular level to prevent the sclerosis of connective tissue sheath. Illustrative examples of vasokinetics and vasodilators are aromatic N-oxides such as: minoxidil, aminexil, triaminodil, pyridine-N-oxide derivatives as described in WO92/21317, 2,6-diamino-derivatives of 4-piperidinopyridine or of 1,3,5-triazine as described in WO91/19701; vasokinetic alkaloids of plant origin such as coumarin, khellin, visnadine, aesculoside, raubasine, vincamine; rubefacients such as nicotinic acid esters (e.g. methyl nicotinate), nicotinic alcohol, pilocarpine, jaborandi, cantharidin, and mentholates (e.g. menthol, menthyl lactate); substances releasing NO in vivo such as: L-arginine, nitroglycerin, isosorbide 5-mononitrate, and isosorbide dinitrate; beta 1-adrenergics (e.g. bamethane sulfate, isoxysuprine); calcium antagonists such as nifedipine, nicardipine, verapamil, diltiazem, nisoldipine, nitrendipine, nilvadipine, isradipine, felodipine, nimodipine, gallopamil, fendiline, flunarizine, amlodipine, diperdipine, fluspirilene, pimozide, fantofarone, nicergoline, and cyclandelate; endo-xanthines (e.g. guanine, adenine); sodium channel agonists such as 1-cyano-2-(1,1-dimethylpropyl)-3-(3-pyridyl)guanidine; ACE-inhibitors such as quinapril, lisinopril, benazepril, captopril, ramipril, fosinopril, cilazapril, and trandolapril.

The “phosphodiesterase inhibitors” counteract the activity of the enzyme that catalyses the conversion of cAMP into 5-AMP (inactive) and/or promote the intracellular accumulation of cAMP, by inhibiting its degradation. Inhibitors of phosphodiesterase comprise in particular “methylxanthines”, such as theophylline, caffeine, pentoxifylline, and propentofylline.

Methylxanthines are also the prototypes of “glycolysis modulators” i.e. substances promoting the metabolic cascade that sustains the follicular protein synthesis during the active phase (anagen).

The “retinoids” have the capacity to increase and regulate the cellular proliferation, to promote the epithelial differentiation and to increase the vascular proliferation in the hair bulb. Retinoic acid is particularly indicated for its ability to increase the number of membrane receptors for EGF without reducing their affinity. Illustrative examples of retinoids include: retinol, retinaldehyde and retinoic acid (tretinoin).

Some of the aforesaid compounds, being particularly preferred for the purposes of the invention, when administered with the UL-OHA of the invention, present a synergistic activity which exceeds the sum of activities of the individual components taken separately. Preferred such compounds are: retinoic acid, minoxidil, finasteride, dutasteride, spironolactone, cyproterone, potassium canrenoate, progesterone, estrone, estrone sulfate, and theophylline.

Further substances optionally usable in combination with UL-OHA are:
peptide growth factors and functional equivalents
endothelial trophic factors

Peptide growth factors (PGF) and functional equivalents are peptides that, at nano- and pico-molar concentrations, favour the proliferation and motility of endothelial cells promoting the neoformation of blood vessels. Useful PGFs for the purposes of the present invention include: VEGF, b,aFGF, EGF, TGF-α,β, PD-ECGF, PDGF, HGF, CSF, G/M-CSF, angiopoietin 1- and -2, IGF-1,2, and PLGF. Other peptides and/or cytokines of the PGF class are: NOS, IL-1,b,6,8, TNF-a, cadherin, vitronectin, fibronectin, fibrin, thrombin, CYR61, CTGF, leptin, PDSF/SF1, HBGFs, CRH, angiogenin (ribonuclease A homologue), and the monomers integrin-αL, -αM and -β2.

The “endothelial trophic factors” include a multiplicity of substances with a reinforcing action toward the endothelial blood vessel, for example: simple plant polyphenols (e.g. cinnamic, benzoic, ferulic, gallic, gentisic, caffeic acids) polycyclic plant polyphenols (e.g. rutin, ruscogenin, escin, esculin, diosgenin, troxerutin, anthocyanins, proanthocyanosides), salvianolic acids, Centella triterpenoids (e.g. asiatic acid, madecassic acid, asiaticoside, madecassoside), phytic acid, ginsenosides (e.g. gibberellic acids and gibberellin-similar hormones), mucopolysaccharides (e.g. dermatan sulfate, chondroitin sulfate, heparan sulfate, natural heparinoids), folic acid, as well as copper peptides and natural and synthetic copper complexes (e.g. Tricomin, Folligen).

Further optional active principles usable in combination with UL-OHAs include: pineal hormones (e.g. melatonin), trichopeptides, lenitive extracts (aloe vera, oat, chamomile), antioxidants (e.g. tocopherol, ascorbates, lipoates, glutathione), plant anti-inflammatories (e.g. allantoin, alpha-bisabolol, azulene, glycyrrhetic acid), carotenoids (e.g. α- and β-carotene), xanthophylls (e.g. lutein, zeaxanthin), tocopherols (e.g. α- and γ-tocopherol) and derivates thereof, alpha hydroxy acids (e.g. glycolic acid, lactic acid, citric acid, their salts and esters), carnosine and urocanic acid, sulfur amino acids (cysteine, cystine, methionine, their amides and salts), as well as anti-dandruff, antiseborrheic, keratolytic and keratoplastic agents (salicylates, sulfur, urea) vitamins, amino acids, etc.

Depending on treatment requirements, the compositions of the invention can contain further pharmaceutical substances such as: antihistamines, local anaesthetics, chemotherapeutic agents, analgesics, narcotics, antivirals, antibiotics, biocides, anti-acne compounds, steroidal and non-steroidal anti-inflammatories.

The UL-OHAs and any of the aforesaid active principles are usable in free form or in the form of inclusion complexes in cyclodextrins and other cross-linked polymers (e.g. cross-linked PVP (XL), dextrans, fullerenes) or as hydrolipid complexes such as microemulsions, liposomes, nanospheres and other systems which may facilitate skin absorption and penetration.

The compositions of the invention may contain any cosmetically and/or pharmacologically acceptable excipients and diluents as well as preservatives (e.g. parabens, idantoin, imidazolidinyl urea, sodium dehydroacetate, benzyl alcohol and quaternary ammonium compounds), silicones, emollients (saturated and unsaturated fatty acids, alcohols and esters, polyols, linear hydrocarbons, paraffins, etc.), isotonic agents, buffers, chelators, hydrocolloids (e.g. polyacrylates, xanthan gum, gelatin, karaya, tragacanth, pectin, sodium carboxymethyl cellulose, hydroxypropyl methylcellulose, hyaluronates, alginates, agar and carrageenans) and all other INCI-CTFA ingredients cited in Annex 93/35/ECC.

The following examples preferentially illustrate the invention and are not intended to limit the scope thereof.

### EXAMPLES

### A) Preparative Examples and Evaluation of Therapeutic Activity

### Example 1

### Preparation of UL-OHA (No. Monosaccharides=2-6) and OHA (No. Monosaccharides=8-24) [Reference Compound]

2.5 g of hyaluronic acid (Connettivina™, Fidia, Italy) is dissolved in 1 litre of a solution of 1M acetate buffer at pH 4.5 and 1.5M sodium chloride.

25 mg of hyaluronidase (600 U/mg, Sigma-Aldrich) are then added and the system is kept under stirring for 4 hours at 37° C.

Monitoring is achieved by means of chromatographic means: a) TLC on silica gel with 1-butanol/AcOH/H₂O 1.5:1:1 or 1:1:1 as eluent, using naphthoresorcinol (0.2% in 96% ethanol, 4% H₂SO₄ at 100° C.) as visualization reagent; b) anion exchange HPLC with YMC-NH₂ (4×250 mm) column with linear gradient of NaH₂PO₄ (from 16 to 800 mM) in 60′ at 1 ml/min at 40° C., with UV detection at 210 nm.

At the end the mixture is heated to 100° C. for 20 minutes, then cooled and centrifuged at 10,000 rpm for 5 minutes. The supernatant is loaded onto an ion exchange column with trimethylammonium ethyl anion groups, of Dowex 1×2 type (mesh size 100-200, phi 1.5×123 cm, Dow Chemical) and eluted with a linear gradient of NaCl solution (from 0.01 M to 0.05 M).

The top fraction containing the UL-OHA (no. of monosaccharides from 2 to 6) is collected separately from the bottom consisting of medium molecular weight OHA (no. of monosaccharides between 8 and 24) then both fractions are desalinized on Sephadex G-10 (Pharmacia, phi 3×124) and lyophilised.

About 0.8 g of UL-OHA and about 0.9 g of OHA are obtained and tested separately for their revascularizing activity.

### Example 2

### Comparative Evaluation of Angiogenic Activity

To determine angiogenic potential the AngioKit (ZHA-1000) from TCS CellWorks (UK) assay can be used a in vitro human model of fibroblasts and endothelial cells growth in extracellular matrix proteins.

The method is carried out by the protocol provided by the manufacturer. Briefly, the wells are treated with a final concentration of 20 μg/ml UL-OHA of the invention (containing 2-6 monosaccharide units) and compared with 20 mg/ml OHA (containing 8 to 24 units), maintained in culture until the eleventh day, then fixed and visualized as described in paragraph 9 of the TCS AngioKit use protocol as issued by the manufacturer.

The capacity of UL-OHAs to induce neo-vascularization compared to OHAs is estimated with a Chalkley graticule is shown in table 1

TABLE 1

Vascular parameter

| | Area (mm²) | Dimension (micron²) |
|---|---|---|
| Placebo | 4.1 ± 0.3% | 165 ± 12 |
| OHA (reference) | 7.2 ± 0.8% | 193 ± 15 |
| UL-OHA | 11.9 ± 1.5% | 274 ± 28 |

The results demonstrate that the activity of UL-OHA in accordance with the invention is far greater than that of the higher molecular weight OHA fraction.

### Example 3

### Evaluation of Increase in Anti-Androgenic Activity

The effect of UL-OHA on inhibiting increase in testosterone 5α-reductase activity can be verified with a micro radioassay.

Biopsies of ventral prostate homogenized with 9 volumes of 0.1 M PBS are filtered through gauze and centrifuged at 3000 rpm for 10 minutes to obtain a crude nuclear fraction. The supernatant is discarded and the pellet resuspended in PBS. This process is repeated once again and the second suspension is used as enzyme solution for the assay.

1 mCi of [4-¹⁴C] testosterone with 17-alpha-estradiol+UL-OHA (calculated for a final conc. of 0.1 mg/ml and 20 μg/ml respectively) and 17-alpha-estradiol (final conc. 0.1 mg/ml) are placed in test tubes and heated to dryness under nitrogen flow. 10 μl of 50 mM NADPH, 60 μl of PBS and 30 μl of testosterone 5α-reductase solution are added and the test tubes and incubated at 37° C. for 1 hour. Finally 0.4 ml of chloroform:methanol (1:2 v/v) are added in order to terminate the reaction and the mixture is centrifuged at 3000 rpm for 10 minutes. A sample from each test tube is applied to silica gel TLC plates and concentrated into a narrow band 3 cm from the bottom by means of 2 brief runs using acetone, then the chromatogram is developed in dichloromethane:diethyl ether (7:1 v/v) followed by chloroform:diethyl ether (9:1 v/v).

The radioactive activity of each extract based on the resulting quantity of testosterone, 5α-dihydro-testosterone, 5α-androstan-3α,17β-diol and 5α-androstan-3β,17β-diol is measured by reading the intensity of isotope emission from the plates as described by Hamada K et al. (IFSCC Magazine 4(2):83-87, 2001). Percentage inhibition of testosterone conversion relative to the control (without substances) indicates an anti-androgenic activity equal to about 30% for the sample with 0.1 mg/ml of 17-alpha-estradiol+20 μg/ml of UL-OHA and equal to 20% for the sample with 0.1 mg/ml of 17-alpha-estradiol.

### Example 4

### UL-OHA Tetrabutylammonium Salt

400 ml of UL-OHA from example 1 are eluted through a column filled with 15 cc of sulfonic resin (Dowex 50×8) in tetrabutylammonium form. The eluate is separated and lyophilized to give 6 g of product.

### Example 5

### UL-OHA Benzyl Ester

1.25 g of UL-OHA tetrabutylammonium salt from example 4 are dissolved in 60 ml of DMSO at 40° C. 0.45 g of benzyl bromide and 0.2 g of tetrabutylammonium iodide are added and the solution is then kept for 12 hours at 35° C. At the end, it is slowly poured into 300 ml of ethyl acetate under agitation. The precipitate is filtered off, washed with 4×50 ml of ethyl acetate and dried under vacuum. 0.9 g of the product of the title are obtained.

### Example 6

### UL-OHA Ethoxycarbonyl-Methyl Ester Sodium Salt

1.25 g of UL-OHA tetrabutylammonium salt from preparative example 4 are dissolved in 60 ml of DMSO at 40° C. 0.3 g of ethyl chloroacetate and 0.2 g of tetrabutylammonium iodide are added and the mixture is stirred for 24 hours at 35° C.

At the end, 7 ml of brine is added and the solution is slowly poured into 300 ml of acetone under stirring. The precipitate is filtered off, washed with 4×50 ml of acetone, and vacuum dried, redissolved in 50 ml of 1% aq. NaCl and poured into 300 ml of acetone under stirring. The new precipitate is filtered off, washed with 50 ml acetone:water 4:1 and with 3×50 ml of acetone then vacuum dried. About 1 g of product is obtained.

### Example 7

### UL-OHA Peracetylated Benzyl Ester

1 g of UL-OHA benzyl ester from preparative example 5 are dispersed in 80 ml of DMF and dissolved under stirring for 2 hours at 50° C.; 100 mg of dimethylamino pyridine and 5 ml of acetic anhydride are then added, and kept under stirring for 48 hours. The solution is vacuum concentrated and precipitated with 150 ml of ether; the precipitate is dissolved in 20 ml of acetone and precipitated with 3×150 ml of ether, then filtered off and dried. 0.8 g of product are obtained.

### B. Formulation Examples

### Examples 8-13

### Hair Cream

| | Ex. 8 | Ex. 9 | Ex. 10 | Ex. 11 | Ex. 12 | Ex. 13 |
|---|---|---|---|---|---|---|
| 100 g of product contain: | (g) | (g) | (g) | (g) | (g) | (g) |
| UL-OHA | 0.50 | 0.50 | 0.50 | 0.50 | 0.50 | 0.1 |
| Estrone sulfate | 0.02 | — | — | — | — | — |
| Minoxidil dihydrochloride | — | 2.0 | 3.0 | — | — | — |
| Azelaic acid | — | 5.0 | — | — | — | — |
| Retinoic acid | — | — | 0.03 | — | — | — |
| Theophylline | 0.3 | 0.3 | — | — | — | — |
| Thyroxine | — | — | — | 0.01 | — | — |
| Melatonin | — | — | — | — | 0.1 | |
| Menthol | 0.5 | 0.5 | 0.5 | — | — | — |
| Pyridoxine | 0.3 | 0.3 | 0.3 | — | — | — |
| Petrolatum | 2.5 | 2.5 | 2.5 | 2.5 | 2.5 | 2.5 |
| Glyceryl monostearate | 3.0 | 3.0 | 3.0 | 3.0 | 3.0 | 3.0 |
| Cetostearyl alcohol | 1.6 | 1.6 | 1.6 | 1.6 | 1.6 | 1.6 |
| POE-(20)-cetyl alcohol | 1.4 | 1.4 | 1.4 | 1.4 | 1.4 | 1.4 |
| Xanthan gum | 0.5 | 0.5 | 0.5 | 0.5 | 0.5 | 0.5 |
| Perfume, preservatives, antioxidants | s.q.(*) | s.q. | s.q. | s.q. | s.q. | s.q. |
| Water q.b. | to 100 | to 100 | to 100 | to 100 | to 100 | to 100 |

(*)sufficient quantity

### Examples 14-19

### Hair Cream-Gel

| | Ex. 14 | Ex. 15 | Ex. 16 | Ex. 17 | Ex. 18 | Ex. 19 |
|---|---|---|---|---|---|---|
| 100 g of product contain: | (g) | (g) | (g) | (g) | (g) | (g) |
| UL-OHA | 0.05 | 0.05 | 0.1 | 0.05 | 0.05 | 0.1 |
| Progesterone | 2.0 | 2.0 | — | — | — | — |
| Minoxidil | 1.5 | 1.5 | 2.0 | — | — | — |
| Theophylline | 0.3 | 0.3 | 0.3 | 0.3 | 0.3 | — |
| Potassium canrenoate | 0.5 | — | — | — | — | — |
| Spironolactone | — | 0.5 | — | — | — | — |
| Menthol | — | 0.3 | — | — | — | — |
| Thyroxine | — | — | — | 0.01 | — | — |
| Melatonin | — | — | — | — | 0.1 | — |
| Cetearyl glucoside/cetearyl alcohol | 1.4 | 1.4 | 1.4 | 1.4 | 1.4 | 1.4 |
| (Emulgade PL 68/50, Henkel) | | | | | | |
| Cetearyl alcohol (Lanette, Henkel) | 0.5 | 0.5 | 0.5 | 0.5 | 0.5 | 0.5 |
| Coco-caprylate (Cedol LC, Henkel) | 1.2 | 1.2 | 1.2 | 1.2 | 1.2 | 1.2 |
| Dicapryl ether (Cetiet, Henkel) | 0.8 | 0.8 | 0.8 | 0.8 | 0.8 | 0.8 |
| Ethyl linoleate/linolenate | 2.0 | 2.0 | 2.0 | 2.0 | 2.0 | 2.0 |
| Petrolatum | 1.5 | 1.5 | 1.5 | 1.5 | 1.5 | 1.5 |
| Sodium alginate | 0.8 | 0.8 | 0.8 | 0.8 | 0.8 | 0.8 |
| Dimethicone (Silicone DC 200CS/Dow) | 0.3 | 0.3 | 0.3 | 0.3 | 0.3 | 0.3 |
| Xylose | 2.5 | 2.5 | 2.5 | 2.5 | 2.5 | 2.5 |
| Preservatives, fragrances | s.q. | s.q. | s.q. | s.q. | s.q. | s.q. |
| Water q.b. | to 100 | to 100 | to 100 | to 100 | to 100 | to 100 |

### Examples 20-25

### Hair Gel

| | Ex. 20 | Ex. 21 | Ex. 22 | Ex. 23 | Ex. 24 | Ex. 25 |
|---|---|---|---|---|---|---|
| 100 g of product contain: | (g) | (g) | (g) | (g) | (g) | (g) |
| UL-OHA | 0.05 | 0.05 | 0.05 | 0.05 | 0.05 | 0.1 |
| Zinc dimethionine | 1.0 | — | — | — | — | — |
| Zinc gluconate | — | 1.0 | — | — | — | — |
| Dermatan sulfate | — | — | 0.5 | — | — | — |
| Green tea catechins (95%) | — | — | 1.0 | — | — | — |
| Triaminoxil | — | — | — | 1.0 | — | — |
| Deanol HCl (Dimethylaminoethanol-HCl) | — | — | — | — | 5.0 | — |
| Glycyrrhetic acid | 0.06 | 0.06 | 0.06 | 0.06 | 0.06 | 0.06 |
| PVP (K 60) | 9.0 | 9.0 | 9.0 | 9.0 | 9.0 | 9.0 |
| Maltodextrin | 6.0 | 6.0 | 6.0 | 6.0 | 6.0 | 6.0 |
| Propylene glycol | 3.0 | 3.0 | 3.0 | 3.0 | 3.0 | 3.0 |
| Hydroxyethyl cellulose | 1.5 | 1.5 | 1.5 | 1.5 | 1.5 | 1.5 |
| Hydrogenated castor oil PEG-40 | 0.3 | 0.3 | 0.3 | 0.3 | 0.3 | 0.3 |
| Disodium EDTA | 0.1 | 0.1 | 0.1 | 0.1 | 0.1 | 0.1 |
| Benzalkonium chloride | 0.5 | 0.5 | 0.5 | 0.5 | 0.5 | 0.5 |
| Xylose | 2.5 | 2.5 | 2.5 | 2.5 | 2.5 | 2.5 |
| Preservatives, fragrances | s.q. | s.q. | s.q. | s.q. | s.q. | s.q. |
| Water q.b. | to 100 | to 100 | to 100 | to 100 | to 100 | to 100 |

### Examples 26-31

### Hair Lotion

| | Ex. 26 | Ex. 27 | Ex. 28 | Ex. 29 | Ex. 30 | Ex. 31 |
|---|---|---|---|---|---|---|
| 100 g of solution contain: | (g) | (g) | (g) | (g) | (g) | (g) |
| UL-OHA | 0.05 | 0.05 | 0.05 | 0.05 | 0.05 | 0.1 |
| Progesterone | 2.0 | — | — | — | — | — |
| Hydrocortisone | — | 1.0 | — | — | — | — |
| Minoxidil | — | — | 1.5 | 1.0 | — | — |
| Theophylline | — | — | 0.5 | — | — | — |
| Serenoa Repens extracts | — | — | — | 2.0 | — | — |
| Azelaic acid | — | — | — | — | 5.0 | — |
| Menthol | 0.5 | 0.5 | 0.5 | 0.5 | 0.5 | — |
| 80/20 cyclosiloxane tetramer/pentamer | 15 | 15 | 15 | 15 | 15 | 15 |
| (SF 1204-GE silicones) | | | | | | |
| Glycerin | 2.5 | 2.5 | 2.5 | 2.5 | 2.5 | 2.5 |
| Ethanol 70° q.b. | to 100 | to 100 | to 100 | to 100 | to 100 | to 100 |

Case Study

A composition as described in formulation example no. 8 was applied to a 42 year old male subject with class II male-pattern baldness for a period of about 6 months.

Hair regrowth was monitored and classified by the usual quali-quantitative trichological parameters with a trichogram, carried out by plucking 50-100 hairs using Klemmer forceps with rubber covered jaws, then cutting the hairs a few centimetres from the proximal end, placing them on the bottom of a Petri dish containing a film of water, observing them with a low magnification microscope and dividing into anagen, catagen and telogen. The phototrichogram was performed under standardized light and distance whereby an area of the scalp of about 1 cm² is photographed, having previously been shaved and determined with three co-ordinates. The area selected from the tip of the nose and the tip of the ears was delineated with a plastic window. In the photograph of the area delineated in this manner the number of hairs were counted. A second photo, taken after 15 to 20 days, enables the number of hairs grown in the anagen phase to be evaluated. By subtraction the number of hairs in telogen is obtained, appearing in the second photo as lighter hair, having not grown or actually disappeared (fallen out). With this method the anagen/telogen ratio in the tested area, the duration of the cycle and the time required to “replace” each telogen (i.e. the duration of telogen itself) can be determined.

The data were integrated with an analysis using optical probe videocapillaroscopy. Significant hair regrowth in the area characterized by alopecia was observed. It was therefore verified that the present invention achieves the aims. It is evident that the compositions and methods of the invention are susceptible to variants which fall within the scope of the inventive concept.

1. Dermatological composition useful for combating hair loss and favouring its regrowth, comprising one or more oligomers of hyaluronic acid and/or esters, salts and solvates thereof, said oligomers consisting of alternated monosaccharide units of D-glucuronic acid and N-acetyl-glucosamine, containing from 2 to 6 of said monosaccharides.

2. Composition as claimed in claim 1, wherein said oligomers are combined with at least a trichogenic agent.

3. Composition as claimed in claim 2, wherein the trichogenic agent is chosen from: testosterone 5-alpha-reductase inhibitors, antagonists of androgen transport, vasokinetics and vasodilators, phosphodiesterase inhibitors and retinoids.

4. Composition as claimed in claim 3, wherein the trichogenic agent is chosen from retinoic acid, minoxidil, finasteride, dutasteride, spironolactone, cyproterone, cyproterone acetate, potassium canrenoate, progesterone, estrone, estrone sulfate, theophylline.

5. Composition as claimed in claim 1, wherein the salts of said oligomers are quaternary ammonium salts.

6. Composition as claimed in claim 1, wherein the esters of said oligomers are chosen from methyl, ethyl, benzyl and ethoxycarbonylmethyl esters.

7. Composition as claimed in claim 1, wherein the concentration of said oligomers is between 0.0001% and 10% by weight on the composition.

8. Composition as claimed in claim 1, wherein the concentration of said oligomers is between 0.01% and 1% by weight on the composition.

9. Composition as claimed in claim 1, wherein the concentration of said oligomers is between 0.01% and 0.1% by weight on the composition.

10. Composition as claimed in claim 1, formulated in a forms suited for topical or transdermal administration.

11. Composition as claimed in claim 1, in the form of a unit doses comprising from 1 to 30 mg of said oligomers.

12. Use of one or more oligomers of hyaluronic acid and/or esters, salts and solvates thereof, said oligomers consisting of alternated monosaccharide units of D-glucuronic acid and N-acetyl-glucosamine, containing from 2 to 6 of said monosaccharides, in the preparation of a drug or a cosmetic composition useful for combating hair loss and favouring its regrowth.

13. Use as claimed in claim 12, wherein said drug also includes at least a trichogenic agent mixed with said oligomers.

14. Use as claimed in claim 12, wherein said drug is useful in the treatment and/or prevention of androgenetic alopecia, alopecia areata, alopecia mucinosa and related disorders.

15. Cosmetic method for combating hair loss and favouring its regrowth, characterised by administering one or more oligomers of hyaluronic acid and/or esters, salts and solvates thereof, said oligomers consisting of alternated monosaccharide units of D-glucuronic acid and N-acetyl-glucosamine, containing from 2 to 6 of said monosaccharides.

16. Method as claimed in claim 15 comprising the administration of trichogenic agent in association with said oligomers.

17. Method as claimed in claim 16, wherein said administration is carried out during a period of between 1 and 50 weeks, with a frequency of between 3 and 14 times per week.
